(19) United States
(12) Patent Application Publication
(10) Pub. No.: US 2019/0358083 A1
(43) Pub. Date: Nov. 28, 2019

(54) APPARATUS FOR EYE TREATMENT

(71) Applicant: Alexander BARASH, Tzoran (IL)

(72) Inventor: Alexander BARASH, Tzoran (IL)

(21) Appl. No.: 16/400,846

(22) Filed: May 1, 2019

(30) Foreign Application Priority Data
May 23, 2018 (IL) 259550

(51) Int. Cl. A61F 9/00

(52) U.S. Cl. CPC A61F 9/0026; A61B 2017/00407

(57) ABSTRACT

An apparatus for treatment of an eye of a patient by a medication stored in an eye drop dispenser is described. The apparatus includes a supporting platform, an upper leg coupled to the supporting platform at one end of the upper leg, a lower leg coupled to the supporting platform at one end of the lower leg and an eye opening assembly configured to pull an upper eyelid up for exposing the eye to the eye drop dispenser and retaining the eye of the patient in the open position. The supporting platform includes a delivery opening configured for holding an eye drop dispenser therein in the upside-down position. The upper leg is configured for positioning on a forehead above an eyebrow of the patient at the other end of the upper leg. The lower leg is configured for positioning on a top part of a cheek below a lower eyelid.

### FIELD OF THE INVENTION

This invention relates to medical techniques for eye rinsing and administering liquid eye medication, and in particular, to an eye treatment apparatus, where eye treatment is assisted by an eye opening assembly.

### BACKGROUND OF THE INVENTION

Human eyes are susceptible to various forms of disease and distress, which may require eye rinsing and/or administration of liquid medications, lubrication drops or other liquid eye treatment agents. People who use contact lenses also tend to use various eye soothing drops to moisten their eyes. Sometimes eye treatment may require regular administration of medications, once or several times during the day.

An eye drop dispenser typically includes an eye drop container or small bottle having resilient walls and a nozzle through which the eye drops are dispensed into the eye.

In order to administer the drops into the eye, a user must tilt his/her head back, position the eye drop dispenser directly over the eye with one hand, open the eyelids with the other hand, and then squeeze the bottle walls.

Such administration, however, can be challenging, especially for children, elderly, and invalids, or for those who have vision problems for various reasons. In particular, it can be difficult to properly position or align the eye drop container to administer eye drops at a particular location in relation to the eye while tilting the head back and simultaneously administering the eye drops. Moreover, since the eyes are typically sensitive and can be subject to a reflex blinking action, when a foreign object approaches the eye lashes and eyelids, the eyelids can sometimes prevent at least some of the eye drops from entering the eye. This can result in inadvertently wasting medicine or, even worse, in inability to administer the correct dosage to the eye. Further, the close positioning of a nozzle of an eye drop dispenser in front of the eye can lead to misjudgment in the distance of the nozzle of the drop container from the eye, and can thereby cause the nozzle to “poke” the exposed eye, and come into contact with the corneal region of the eye. This can potentially cause or increase the likelihood of infection, irritation, or even injury to the eye.

A number of eye drop treatment devices have been proposed in the art that attempt to remedy the problems related to self-administration of eye-drops.

For example, U.S. Pat. No. 6,090,086 describes an eye drop applicator which has an adjustable guide arm. The guide arm defines a semi-rigid member attached to an eye drop container. The guide arm serves as a point of tactile reference to the user who seeks to dispense liquid eye medicament. It further serves as a retractor of the lower eyelid, thereby allowing the user to place eye drops accurately and efficiently into the conjunctiva. The eye drop applicator can be manipulated with one hand, and avoids the necessity of the user placing his or her fingers near the eye socket, thereby maximizing the sterility and effectiveness of the medicament.

U.S. Pat. No. 9,033,941 discloses a system and apparatus for improving the accuracy with which a person may apply eye drops to their own eye. The apparatus holds a bottle of eye drops at a fixed and certain distance above the eye, beside the nose, and below the forehead brow such that the user may easily apply eye drops consistently to one particular portion of their own eye. The apparatus sits on the nose using a nasal bridge device and also rests on two pillars that are placed on the user's forehead.

U.S. Pat. No. 9,610,192 describes an eyedrop bottle holder with resilient arms formed from an inverted U-shaped band made from resilient injection molded plastic. The top middle portion of the inverted U-shape band includes a centrally located opening through which the top (dispensing portion) of a standard eyedrop bottle containing eyedrop solution may be positioned and held. The right and left arms of the U-shape band each terminate in an outwardly disposed J-shape foot, the underside of which is covered by a soft rubber-like pad. When a user inserts a standard eyedrop bottle into the holder, the dispensing tip of the eyedrop bottle may be positioned in close proximity to the user's eye. The user may cause his or her eye lid to remain open by using the fingers of one hand to squeeze the right and left arms of the holder together, and then places the pad of one arm on the upper ridge of the orbital eye socket, and the pad of the second arm on the lower ridge of the orbital eye socket. Releasing the arms causes the skin of the user's upper and lower eyelids to be spread apart from each other and remain spread during an eyedrop solution dispensing event.

U.S. Pat. Appl. Publ. No. 2015/088099 describes an eye drop assist apparatus and method for sequentially administering an ocular medication into patient's eyes. A bottle or vial containing liquid eye drops is supported in a holder. First and second nasal locators extend in opposite directions from the holder. The nasal locators are separated by the pupillary distance, with the dispensing tip of the bottle located mid-way between. A person administering eye drops abuts the first nasal locator against the top of the patient's nasal bridge while aligning the tip of the bottle directly over a first eye before squeezing the bottle to forcibly expel one or more drops into the first eye. The person then linearly translates the bottle so that the second nasal locator abuts against the top of the patient's nasal bridge while aligning the tip of the bottle directly over a second eye. The bottle is then squeezed again to forcibly expel drops into the second eye.

U.S. Pat. No. 9,545,333 describes an eye dropper positioning and guiding apparatus that includes a resilient guiding device having a selectively compressible member having a first opening adapted to receive an eye drop applicator and a second opening adapted to position an eye within an area of the second opening to receive a fluid from the eye drop applicator. The selectively compressible member is adapted for selective linear and nonlinear movement in relation to the eye positioned within the area of the second opening.

### SUMMARY OF THE INVENTION

Despite known techniques in the area of eye treatment, there is still a need for a novel apparatus for eye rinsing and administering liquid eye medication that can be used in two related areas, such as clinical use and everyday use by any user, including children, impaired and elderly patients. In both these areas, opening and retention of the eye during treatment is required in order to render them easy to use and cost effective, so that these eye treatment apparatuses may be deployed to a large population.

It would be useful to provide an eye treatment apparatus, which allows for dispensing of liquid ocular medication in such a manner that the user need not place his/her fingers near the surface of the eyelids.

It would also be useful to provide an eye treatment apparatus, which allows for positioning or guiding the eye drop dispenser to the desired position relative to the eye.

It would also be advantageous to provide an eye treatment apparatus, which can be adapted to work with eye drop dispensers having containers and nozzles of any non-uniform design, size, or shape or nozzles of varying lengths.

It would also be advantageous to provide an eye treatment apparatus, in which a nozzle of the dispensing container would be fixed and located at a desired distance from the eye, so as to avoid the likelihood that the nozzles that dispense the eye drops would come in direct contact with the eye, thus avoiding the possibility of causing damage to the eye.

It would still be advantageous to provide an eye treatment apparatus, that can be used by individuals having different facial features such as different width and height of the nose, or individuals having bulging eyes or eyes below a relatively normal position, thus avoiding the possible difficulty or inability in achieving an acceptable position of an eye drop apparatus in relation to an eye, prior to dispensing the medication.

The present invention satisfies the aforementioned needs in the art by providing a novel apparatus for treatment of an eye of a patient by a medication stored in an eye drop dispenser. The apparatus enables a user to place the apparatus on his/her face, and to align and stabilize an eye drop dispenser. The apparatus is equipped with an eye opening assembly enabling the treated eye to remain open while applying eye drops or rinsing medication to the eye in a relatively easy manner. A user of the apparatus may be either the patient himself or a caregiver.

According to an embodiment of the present invention, the eye treatment apparatus includes a supporting platform including a delivery opening configured for holding an eye drop dispenser therein in the upside-down position.

According to an embodiment of the present invention, the supporting platform has a top side, an under side, and a lateral side. In turn, the lateral side has a right wall, a left wall, an upper wall and a lower wall.

According to an embodiment of the present invention, the delivery opening has a prismatic shape formed by inner surfaces of the platform connecting the upper, left and lower walls of the supporting platform and a grasping claw projecting from an inner surface of the right wall of the supporting platform. The grasping claw, for example, has a cylindrical concave end.

According to an embodiment of the present invention, the eye treatment apparatus further includes a restricting member. The restricting member is configured to limit insertion of the eye drop dispenser into the delivery opening of the supporting platform for holding the eye drop dispenser at a selected distance above the eye of the patient in order to prevent direct contact of the dispenser with the eye. The restricting member includes a through opening having a predetermined dimension adapted to match the dimension of a nozzle of the eye drop dispenser.

The eye treatment apparatus of the present invention also includes a locking mechanism integrated with the supporting platform, and is configured to provide secure grasping of the eye drop dispenser within the delivery opening.

According to one embodiment of the present invention, the locking mechanism includes a ratchet mechanism arranged under the lower wall, however other locking mechanisms, such as a screw clamp, cam clamp, eccentric clamp, etc. are also contemplated.

According to an embodiment, the ratchet mechanism includes a rack plate and a pawl plate. The rack plate is an extension of the left wall from the corner of the intersection of the left wall with the lower wall. In turn, the pawl plate is an extension of the right wall from the corner of intersection of the right wall with the lower wall.

According to another embodiment of the present invention, the locking mechanism includes a ratchet mechanism arranged above the upper wall. In this embodiment, the ratchet mechanism includes a rack plate which is an extension of the right wall from the corner of the intersection of the right wall with the upper wall, and a pawl plate which is an extension of the left wall.

The rack plate includes a tooth portion having ratchet teeth located at the inner surface of the rack plate. The pawl plate includes a pawl tooth arranged at an end of the pawl plate and is configured for engagement with the ratchet teeth.

According to yet another embodiment of the present invention, the delivery opening has a cylindrical shape formed by the inner walls of the supporting platform. The cylindrically shaped supporting platform includes an elongated vertical slit arranged at the lateral side of the supporting platform. The slit extends from the top side to the under side of said supporting platform.

According to one embodiment of the present invention, the supporting platform is formed of one or more resilient materials. This enables expansion and contraction of the supporting platform for varying the cross-sectional dimensions of the delivery opening upon engaging with the eye drop dispenser to secure it within the delivery opening.

According to another embodiment of the present invention, the supporting platform further includes a supporting member. The supporting member is connected to the lateral side of the supporting platform. The supporting member is adapted to secure the eye drop dispenser within the delivery opening.

The eye treatment apparatus also includes an upper leg and a lower leg coupled to the supporting platform at one end of the upper and lower legs. The upper leg is configured for positioning on a forehead above an eyebrow of the patient at the other end of the upper leg, while the lower leg is configured for positioning on a top part of a cheek below a lower eyelid. Thus, pressure applied to the apparatus during eye treatment procedure is applied to the patient's face, rather than to the eye under treatment.

According to an embodiment of the present invention, the upper and lower legs are terminated in feet configured for positioning on the forehead and the cheek of the patient, correspondingly. Each foot can, for example, be integrated with the corresponding leg in one unit. Alternatively, the feet can be separate elements pivotally attached to the ends of the upper and lower legs, correspondingly. When desired, the feet can include cushion pads.

According to an embodiment of the present invention, the lower leg includes a bending mechanism located between the supporting platform and the corresponding foot. The bending mechanism is configured to allow the lower leg to be bent into angular form to limited angles having predetermined values outwardly or inwardly with respect to a face of the patient. This feature can limit pressure on the lower eyelid when the apparatus is pushed towards the face, especially when the upper eyelid is opened.

According to one embodiment of the present invention, the bending mechanism divides the lower leg into two leg parts, which are manufactured as separately assembled pieces, and connected by an elastic member passing within grooves formed at meeting ends of the leg parts, correspondingly. The leg parts are separated from each other by gaps formed at two sides of the elastic member.

According to one embodiment of the present invention, the upper leg and the lower leg are manufactured separate from the supporting platform, and are assembled together with the platform by connecting the upper leg and the lower leg to the upper wall and the lower wall of the platform, correspondingly.

According to another embodiment of the present invention, the supporting platform, the upper leg and at least a portion of the lower leg, are manufactured as one entire part.

According to an embodiment of the present invention, the eye treatment apparatus includes an eye opening assembly configured to pull an upper eyelid up for exposing the eye to the eye drop dispenser, and for retaining the eye of the patient in its open position as long as is required for eye treatment. The eye drop dispenser includes a container for holding medicament and a nozzle extending from the container and allowing the medicament to be ejected therefrom. The eye drop dispenser is aligned such that an alignment axis passing through the nozzle of the eye drop dispenser also passes through the eye.

According to an embodiment of the present invention, the eye opening assembly includes an eyelid pulling frame including two frame arms having distal ends and separated at a predetermined distance. The eye opening assembly also includes an eyelid-retaining thread stretched between the distal ends of the frame arms. The eyelid-retaining thread is a flexible filament having a predetermined strain. The eyelid-retaining thread is configured to take an outer contour of the eyeball when the eyelid-retaining thread is placed on the upper eyelid by applying a predetermined pressing force against the eye.

The eye opening assembly also includes a spring member configured to provide the predetermined pressing force to the eyelid pulling frame. According to an embodiment of the present invention, the spring member includes a strip spring.

According to an embodiment of the present invention, one end of the spring member is connected to the pulling frame, while the other end of the spring member is connected to the distal end of the upper leg.

According to another embodiment of the present invention, the spring member is connected to the pulling frame at its one end and to the supporting platform at its other end.

There has thus been outlined, rather broadly, the more important features of the invention in order that the detailed description thereof that follows hereinafter may be better understood. Additional details and advantages of the invention will be set forth in the detailed description, and in part will be appreciated from the description, or may be learned by practice of the invention.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1A illustrates an elevated perspective view of an apparatus for eye treatment, according to an embodiment of the present invention;

FIG. 1B illustrates an elevated perspective view of an apparatus for eye treatment, according to another embodiment of the present invention;

FIG. 1C illustrates an elevated perspective view of an apparatus for eye treatment, according to a further embodiment of the present invention;

FIG. 2 illustrates a perspective view of the apparatus for eye treatment of FIG. 1A in use;

FIGS. 3A, 3B and 3C illustrate an example of a bending mechanism of the lower leg of the apparatus of FIGS. 1A and 1B, and operation of the bending mechanism, according to an embodiment of the present invention;

FIG. 4 illustrates a side view of an apparatus for eye treatment, according to still another embodiment of the present application;

FIGS. 5A-5C illustrate a method of use of the apparatus of FIG. 1A, according to an embodiment of the present invention;

FIGS. 6A-6E illustrate a method of use the apparatus of FIG. 1A, according to another embodiment of the present invention;

FIG. 7 illustrates an elevated perspective view of an apparatus for eye treatment, according to another embodiment of the present invention; and

FIG. 8 illustrates an elevated perspective view of an apparatus for eye treatment, according to a further embodiment of the present invention.

### LIST OF REFERENCE NUMERALS

10, 100, 1000, 3000, 4000—eye treatment apparatus;

11—supporting platform;

110—lateral side;

111—top side;

112—under side;

113—right wall;

114—left wall;

115—upper wall;

116—lower wall;

117—inner surfaces of the platform 11;

118—inner surface of the right wall 113;

12—delivery opening;

13—eye;

134—upper eye lashes;

135—groove;

131—eyebrow;

132—lower eyelid;

133—upper eyelid;

14—grasping claw;

140—cylindrical concave end;

20—locking mechanism;

200, 2000—ratchet mechanism;

15, 150—rack plate;

151—inner surface of the rack plate 15;

153—ratchet teeth;

152—tooth portion;

16, 160—pawl plate;

17—upper leg;

171—foot of the upper leg 17;

18—lower leg;

181—foot of lower leg 18;

182—bending mechanism;

183, 184—parts of the lower leg 18;

185—elastic member;

187,188—gaps between parts 183,184;

1830, 1840—grooves in parts 183,184;

19—eye opening assembly;

191—eyelid pulling frame;

192,193—frame arms;

194—eyelid-retaining thread;

195—spring member;

1920, 1930—distal ends of the frame arms 192, 193;

1952—one end of spring member 195;

1951—other end of spring member 195;

21—face;

22—patient;

23, 300—eye drop dispenser;

231—sides of cylindrical portion of eye drop dispenser;

232—nozzle of eye drop dispenser;

24—forehead;

25—cheek;

500—restricting member;

510—through opening;

600—supporting member;

610—supporting inner wall;

620—supporting outer wall;

70—elongated vertical slit.

DETAILED DESCRIPTION OF EMBODIMENTS

The principles and operation of the eye treatment apparatus for eye rinsing and administering liquid eye medication according to the present invention may be better understood with reference to the drawings and the accompanying description, it being understood that these drawings and examples in the description are given for illustrative purposes only and are not meant to be limiting. The same reference numerals and alphabetic characters will be utilized for identifying those components which are common in the eye treatment apparatus and its components shown in the drawings throughout the present description of the invention.

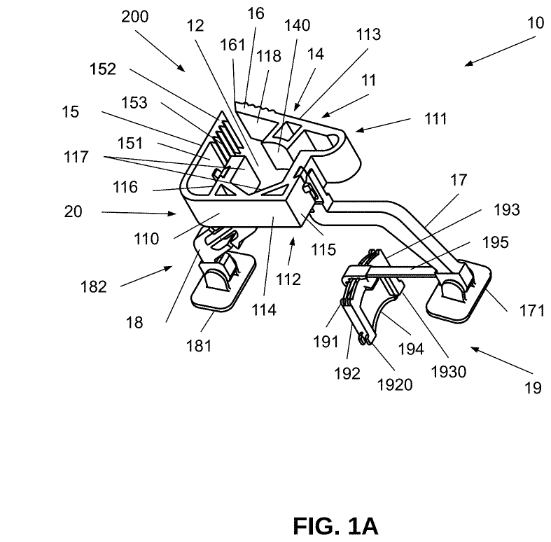

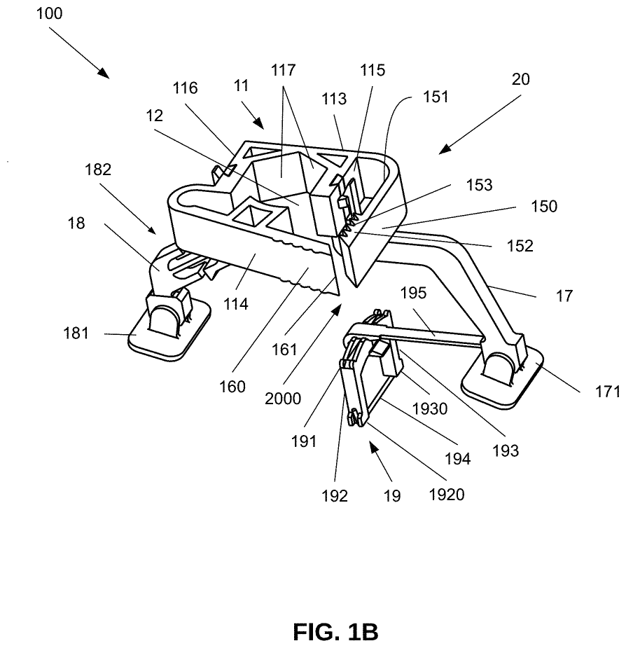

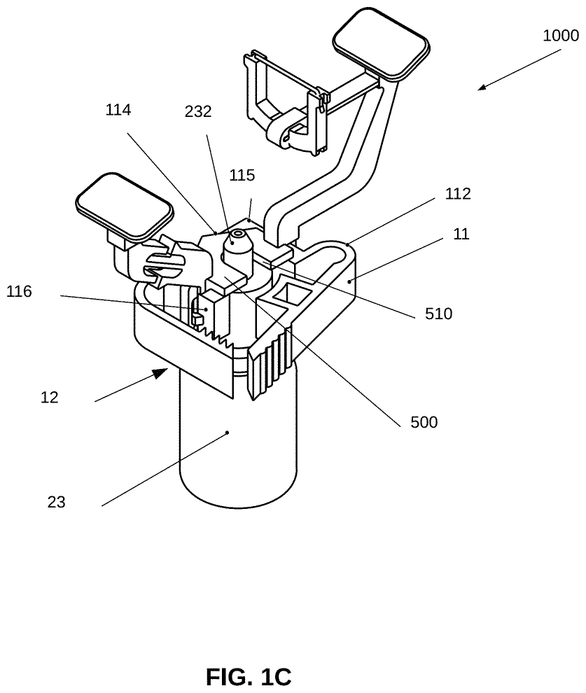

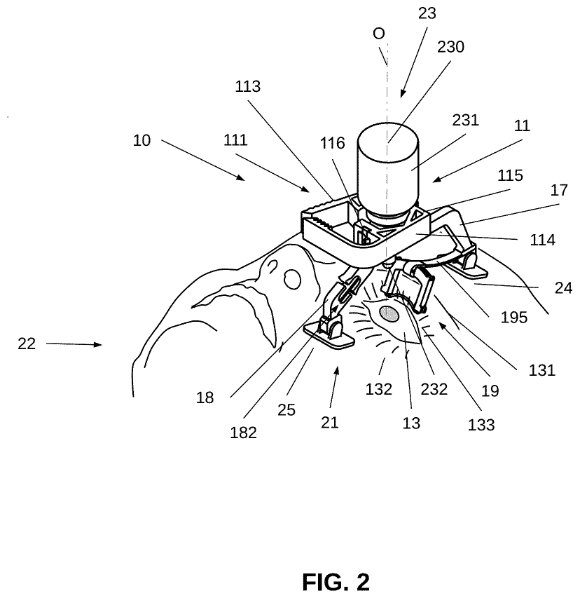

Referring to FIGS. 1A and 2 together, an elevated perspective view of an apparatus 10 for eye treatment, is illustrated in FIG. 1A, according to one embodiment of the present invention. In turn, FIG. 2 illustrates an elevated perspective view of the eye treatment apparatus 10 of FIG. 1 in use, where the eye treatment apparatus 10 is positioned on a face 21 of a patient 22.

The eye treatment apparatus 10 includes a supporting platform 11 configured for holding an eye drop dispenser 23. The supporting platform 11 has a top side 111, an under side 112, and a lateral side 110 having a right wall 113, a left wall 114, an upper wall 115 and a lower wall 116. The terms “top side”, “under side”, “lateral side”, “right wall”, “left wall” “upper wall” and “lower wall” are used herein for the purpose of description of a relationship between the different parts of the supporting platform 11 of the eye treatment apparatus 10 and a face 21 of the patient (22 as shown in FIG. 2), rather than for description of orientation of the eye treatment apparatus 10 in space.

The supporting platform 11 includes a delivery opening 12 configured for holding the eye drop dispenser 23 in the upside-down position in the delivery opening 12. The eye treatment apparatus can be used with different types of eye drop dispensers. According to an embodiment of the present invention, the eye drop dispenser 23 includes a container in the form of small cylindrical bottle 230 configured for holding medicament. Such an eye drop bottle can be of any size or shape that allows it to be placed in the delivery opening 12 of the supporting platform 11 in the upside-down position. At the top of the eye drop dispenser 23 is a nozzle 232 that extends from the cylindrical portion forming sides 231 of the dispenser 23, and allows liquid medicament to be ejected therefrom. The eye drop bottle of the dispenser 23 is usually fabricated from a pliable and resilient material, such as plastic, that allows the user to squeeze liquid from the bottle by squeezing the sides 231 of the eye drop dispenser 23 by the fingers, thus causing the medication to fall from the nozzle of the eye drop dispenser into the eye 13.

The delivery opening 12 may have different shapes to accommodate to the shape of the walls of the eye drop dispenser 23. According to the embodiment shown in FIG. 1A, the delivery opening 12 has a prismatic shape formed by inner surfaces 117 of the platform 11 and a grasping claw 14. The inner surfaces 117 connect the upper, left and lower walls 115, 114 and 116. The grasping claw 14 projects from an inner surface 118 of the right wall 113 of the supporting platform 11. According to an embodiment, the grasping claw 14 has a cylindrical concave end 140 to accommodate to the cylindrical shape of the eye drop dispenser 23, however other configurations are also contemplated. It should be understood that the configuration of the delivery opening 12 shown in FIG. 1A can be used with various dispensers that may have different diameters of the cylindrical portion.

In order to provide secure grasping of the eye drop dispenser 23 within the delivery opening 12, the eye treatment apparatus 10 includes a locking mechanism 20 integrated with the supporting platform 11. According to the embodiment shown in FIG. 1A, the locking mechanism 20 includes a ratchet mechanism 200 arranged under the lower wall 116.

The ratchet mechanism 200 includes a rack plate 15 and a pawl plate 16. The rack plate 15 is an extension of the left wall 114 from the corner of the intersection of the left wall 114 with the lower wall 116, while the pawl plate 16 is an extension of the right wall 113. The rack plate 15 includes a tooth portion 152 having ratchet teeth 153 located at the inner surface 151 of the rack plate 15. On the other hand, the pawl plate 16 includes a pawl tooth 161 arranged at an end of the pawl plate 16 and configured for engagement with the ratchet teeth 153. In operation, the user inserts the eye drop dispenser 23 into the delivery opening 12 upside-down, and then bends the pawl plate 16 towards the rack plate 15 until the pawl tooth 161 at the end of the pawl plate 16 mates up with the toothed portion 152 of the rack plate 15.

According to an embodiment of the present invention, the supporting platform 11, the rack plate 15 and the pawl plate 16 are integrated together, and are formed from a light, resilient and pliable material. An acceptable material for the supporting platform 11, the rack plate 15 and the pawl plate 16, such as plastic, can, for example, be used. The rack plate 15 and the pawl plate 16 should have relatively low toughness to allow the user to bring the pawl plate 16 to the rack plate 15 by his/her fingers in order to lock the ratchet locking mechanism 13, and thereby to secure the eye drop dispenser 23 within the delivery opening 12 of the supporting platform 11 by providing a gripping force acting on the eye drop dispenser 23.

It should be noted that although in FIG. 1A the locking mechanism 13 is shown under the lower wall 116, other configurations for securing the eye drop dispenser 23 within the delivery opening 12 are also contemplated.

For example, FIG. 1B illustrates an elevated perspective view of an apparatus 100 for eye treatment, according to another embodiment of the present invention. According to the embodiment shown in FIG. 1B, the locking mechanism 20 includes a ratchet mechanism 2000 arranged above the upper wall 115.

As shown in FIG. 1B, the ratchet mechanism 2000 includes a rack plate 150 and a pawl plate 160. The rack plate 150 is an extension of the right wall 113 from the corner of the intersection of the right wall 113 with the upper wall 115, while the pawl plate 160 is an extension of the left wall 114. The rack plate 150 includes a tooth portion 152 having ratchet teeth 153 located at the inner surface 151 of the rack plate 150. On the other hand, the pawl plate 160 includes a pawl tooth 161 configured for engagement with the ratchet teeth 153. In operation, the user inserts the eye drop dispenser 23 into the delivery opening 12 upside-down, and then bends the pawl plate 160 towards the rack plate 150 until the pawl tooth 161 at the end of the pawl plate 160 mates up with the toothed portion 152 of the rack plate 150.

Referring to FIG. 1C, an elevated perspective view of an apparatus 1000 for eye treatment is illustrated, according to a further embodiment of the present invention. The apparatus 1000 differs from the apparatus (10 in FIG. 1A) in the fact that it further includes a restricting member 500 configured to limit insertion of the eye drop dispenser 23 into the delivery opening 12 of the supporting platform 11. This provision allows holding the eye drop dispenser 23 at a selected distance above the eye 13 of the patient in order to prevent direct contact of the dispenser with the eye.

The restricting member 500 is connected to the under side 112 of the upper, left and lower walls 115, 114 and 116 of the supporting platform 11.

Connection of the restricting member 500 to the platform 11, can for example, be made by using a suitable fastener or connector. Alternatively, the supporting platform 11 and the restricting member 500 can be manufactured as one entire part.

The restricting member 500 includes a through opening 510 having a predetermined dimension adapted to match the dimension of the nozzle 232 of the eye drop dispenser 23. The restricting member 500 is adapted to prevent direct contact of the tip of the nozzle 232 with the eye. According to the embodiment shown in FIG. 1C, the through opening 510 has a U-shape, however, generally, the through opening 510 may have any other shape and dimension to accommodate to the shape and size of the nozzle 232 of the eye drop dispenser 23.

Referring to FIGS. 1A, 1B and 2 together, the eye treatment apparatus 10, 100 also includes an upper leg 17 and a lower leg 18 which are coupled to the supporting platform 11 at their ends. The upper leg 17 and a lower leg 18 can be positioned on the face of the patient 22 at their other ends to support the supporting platform 11 on the face over the patient's eye 13. As shown in FIG. 2, the upper leg 17 is positioned on the forehead 24 of the patient 22 above an eyebrow 131, while the lower leg 18 is positioned on a top part of the cheek 25 below a lower eyelid 132. In operation, an alignment axis 0 passing through the nozzle 232 of the eye drop dispenser 23 should also pass through the eye 13.

According to an embodiment of the present invention, the upper leg 17 and the lower leg 18 are terminated in feet 171 and 181, correspondingly. When desired, the feet 171 and 181 include cushion pads. The cushion pads can, for example, be made from polyester foam or ribbon.

Preferably, but not mandatory, the feet 171 and 181 are separate elements that are pivotally attached to the ends of the upper and lower legs 17 and 18, correspondingly, for example, by using hinges (not shown). The cushion pads of the feet 171 and 181 should have surface contact area with the patient's face sufficient for the cushion pads to gently engage with the skin of the patient, and should not cause significant discomfort to the patient during eye treatment procedures. For example, the surface contact area of the cushion pads of the feet 171 and 181 can be in the range of 3 cm² to 6 cm².

To further facilitate the accurate administration of eye drops, the tactile surface of cushion pads of the feet 171 and 181 with the face of the patient 22 can be rough. This provision can provide desired friction between the tactile surface and the cheek 25 of the patient 22 and assist the lower leg 18 in retracting the lower eyelid so as to expose the conjunctiva. In this manner, greater control over the position of the dispenser 23 can also be maintained by the user of the apparatus 10, 100. The method of operation of the eye treatment apparatus 10, 100 is shown herein below in detail.

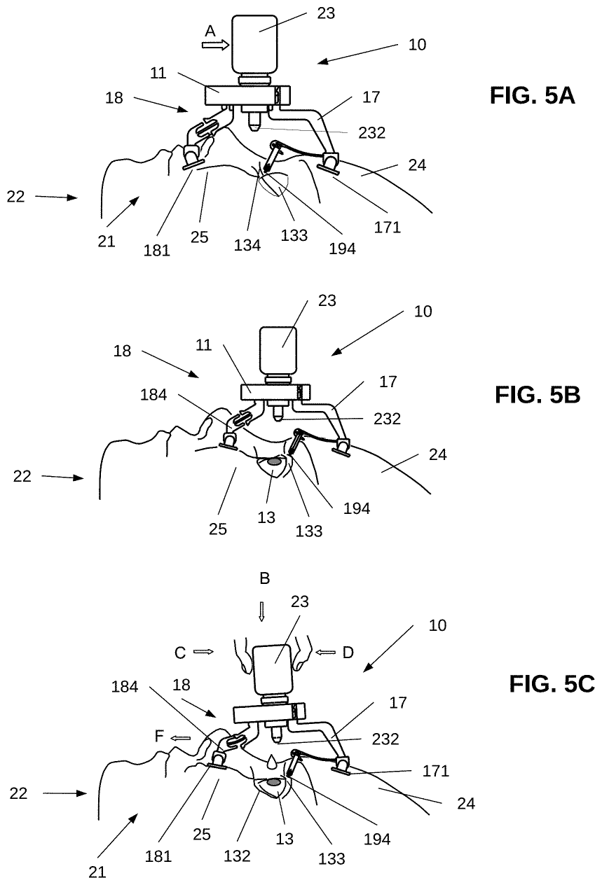

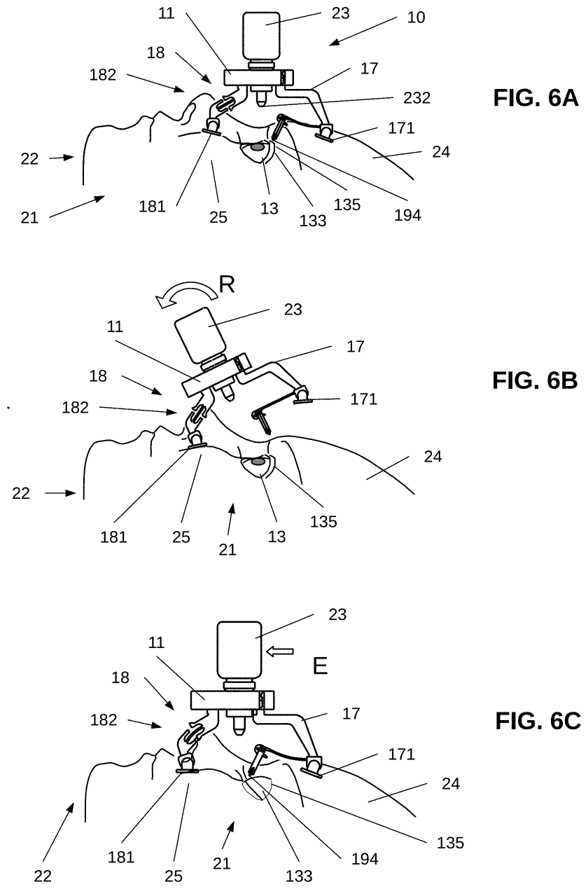

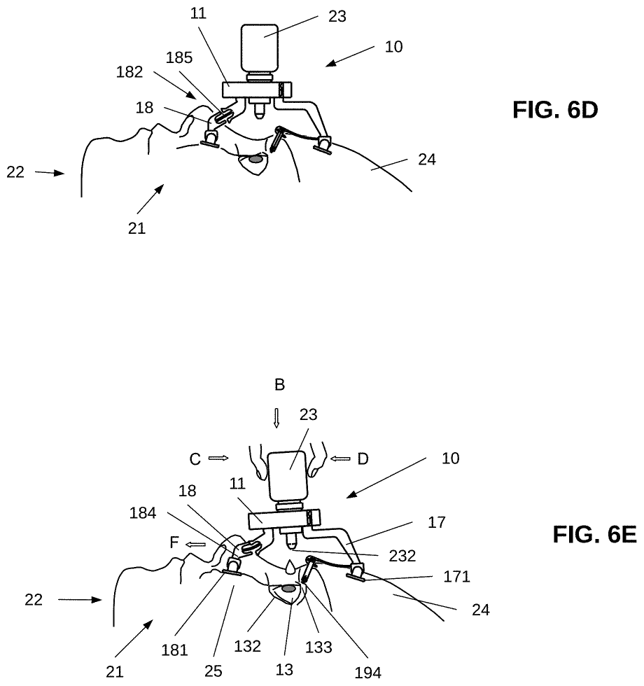

According to an embodiment of the present invention, the lower leg 18 includes a bending mechanism 182 located between the supporting platform 11 and the foot 181. The bending mechanism 182 allows the lower leg 18 to be bent into angular form to limited angles having predetermined values outwardly or inwardly with respect to the patient's face. As will be described herein below in detail with reference to FIGS. 5C and 6E, when the lower leg 18 bends inwardly, the patient's cheek 25 is pulled down together with the lower eyelid 132 for opening a conjunctival sac of the eye 13. It should be noted that opening of the lower eyelid 132 allows, when required, administration of the medication into the conjunctival sac, rather than onto more sensitive parts of the eye, such as the cornea.

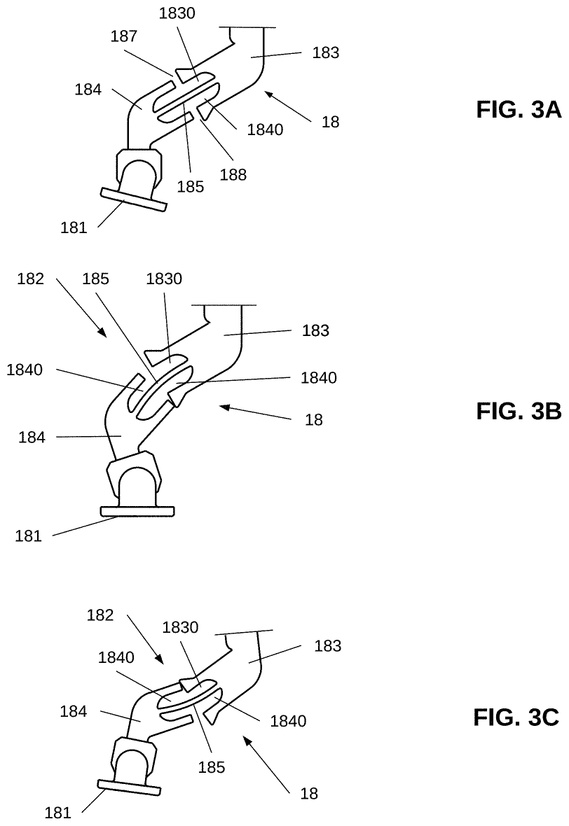

Referring to FIG. 3A, 3B and 3C together, an example of the bending mechanism 182 and its operation is illustrated, according to an embodiment of the present invention. According to this embodiment, the bending mechanism 182 divides the lower leg 18 into two leg parts 183 and 184, which are manufactured as separately assembled pieces, and connected by an elastic member 185 passing within grooves 1830 and 1840 formed at meeting ends of the leg parts 183 and 184, correspondingly.

The elastic member 185 can be formed as an integral part of the lower leg 18 together with the leg parts 183 and 184, as shown in FIG. 3A, 3B and 3C. Alternatively, the elastic member 185 can be a separate unit coupled to the leg parts 183 and 184. In this case, the elastic member 185 may be made either from the same material as the leg parts 183 and 184 or from a different material.

As illustrated in FIG. 3A, when the leg 18 is straight, the leg parts 183 and 184 are separated from each other by gaps 187 and 188 formed at two sides of the elastic member 185. FIGS. 3B and 3C show the states when the leg 18 is bent outwardly and inwardly, correspondingly, to maximal bending angles.

According to the embodiments shown in FIGS. 1A, 1B and 2, the upper leg 17 and the lower leg 18 are manufactured separately from the supporting platform 11 and assembled together with the platform 11 by connecting the upper leg 17 and the lower leg 18 to the upper wall 115 and the lower wall 116 of the platform 11, correspondingly. Connection of the upper leg 17 and the lower leg 18 to the platform 11, can for example, be made by using a suitable fastener or connector.

According to another embodiment of the present invention, the supporting platform 11, the upper leg 17 and the lower leg 18 are manufactured as one entire part.

The supporting platform 11, the upper leg 17 and the lower leg 18 of the eye treatment apparatus 10, 100 can be formed from a light and pliable material. An example of a material suitable for the present invention includes, but is not limited to, plastic.

The dimensions of the supporting platform 11, the upper leg 17 and the lower leg 18, and the distance between the feet 171 and 181, is in proportion to the typical dimensions of a human face. When desired, a set of the upper leg 17 and the lower leg 18 having various dimensions can be manufactured and used with the supporting platform 11 to match diverse face sizes of patients. These various size legs 17 and 18 may be provided separately from the supporting platform 11 in order to allow users to select the legs with the proper size, and then attach the proper size legs to the supporting platform 11. Thus, the supporting platform 11 may be available for purchase with a plurality of legs 17 and 18 having different sizes in order to accommodate a plurality of users for the same apparatus, for example, children and parents in a family.

Turning back to FIGS. 1A, 1B and 2, the eye treatment apparatus 10, 100 also includes an eye opening assembly 19 configured to pull an upper eyelid 133 up for exposing the eye 13 to the nozzle 232 of the eye drop dispenser 23, and for retaining the eye 13 in the open position as long as required for administration of medication. According to the embodiment shown in FIGS. 1A, 1B and 2, the eye opening assembly 19 includes an eyelid pulling frame 191 including two frame arms 192 and 193 having distal ends 1920 and 1930 separated at a predetermined distance. For example, the predetermined distance between the distal ends 1920 and 1930 can be in the range of about 10 mm to 18 mm that corresponds to a value of about half to two-thirds of a horizontal size of an open eye of a patient (child or adult). As shown in FIGS. 1A, 1B and 2, the eyelid pulling frame 191 has a U-type shape, however, it can also have a V-type shape or any other suitable shape.

The eye opening assembly 19 also includes an eyelid-retaining thread 194 stretched between the distal ends 1920 and 1930 of the frame arms 192 and 193. According to an embodiment, the eyelid-retaining thread 194 is a flexible filament having a predetermined strain that can follow an outer contour of the eyeball and conform to the shape of the eyeball when the eyelid-retaining thread 194 is placed on the upper eyelid 133 by applying a predetermined pressing force against the eye 13 along the line of contact of the eyelid-retaining thread 194 with the upper eyelid 133. For example, the predetermined pressing force can be in the range of about 0.5N (Newton) to 1.5N. Examples of materials suitable for the eyelid-retaining thread 194 include, but are not limited to, silicone, rubber and other flexible materials. According to an embodiment, the eyelid-retaining thread 194 can also be in the form of a thin coil spring.

In order to provide such pressing force, the eye opening assembly 19 of the eye treatment apparatus 10, 100 includes a spring member 195 configured to provide the predetermined pressing force to the eyelid pulling frame 191. In operation, the pressing force provided by the spring member 195 is transferred to the upper eyelid via the eyelid-retaining thread 194 in order to hold the upper eyelid open when the eyelid-retaining thread 194 is placed thereon.

According to the embodiments shown in FIGS. 1A, 1B and 2, the spring member 195 is in the form of a straight strip spring that can, for example, be made from a spring steel material, however use of other materials and implementations of the spring member 195 are also contemplated. For example, the spring member 195 can be in the form of a spiral spring, a rod, etc.

According to the embodiments shown in FIGS. 1A, 1B and 2, a length and configuration of the strip spring is such that a distance between the eyelid-retaining thread 194 and the alignment axis (O in FIG. 2) passing through the nozzle 232 of the eye drop dispenser 23 and the eye 13 has a predetermined value. This value can, for example, be in the range of about 5 mm to 12 mm.

As shown in FIGS. 1A, 1B and 2, one end of the strip spring member 195 is connected to (e.g., hooked to) the pulling frame 191, while other end of the strip spring member 195 is connected to the distal end of the upper leg 17 by using a suitable connector.

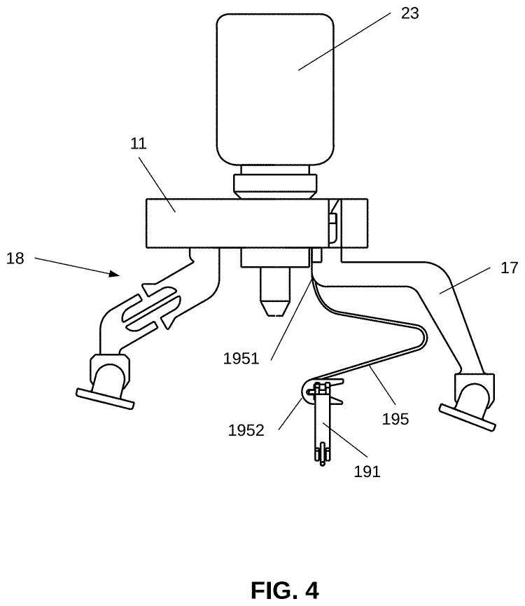

According to another embodiment of the present invention, as shown in FIG. 4, the spring member 195 can be connected to the pulling frame 191 at its one end (indicated by reference numeral 1952) and to the supporting platform 11 at its other end (indicated by reference numeral 1951).

The method of operation of the eye treatment apparatus 10, 100 is shown herein below in detail.

Generally, a user of the eye treatment apparatus 10 may be either the patient 22 himself/herself, or a caregiver. The caregiver hereinafter will also be referred to as an operator of the apparatus 10. The apparatus of the present invention is adapted to administer eye drops in an eye 13 of the patient 22 in an easy manner without wasting eye drops or aggravating the patient.

First, the user (patient or caregiver) inserts an eye drop dispenser 23, such as an eye drop bottle or a bottle with a rinsing liquid, upside-down into the delivery opening 12 of the supporting platform 11, and fixes it by using the locking mechanism 20. The eye treatment apparatus 10 is now ready for use and may be either immediately placed on the user's face, or stored with a nozzle cap placed on the bottle of the dispenser 23 while it is in the platform 11.

Referring to FIGS. 5A through 5C, a method of administration of medication into an eye of a patient by using the apparatus 10 by an operator (not shown) is illustrated, according to an embodiment of the present invention. Although treatment of the patient's left eye is shown in FIGS. 5A-5C, generally, the eye can be either the patient's left eye or right eye, mutatis mutandis. Although the user (i.e., operator) can hold the apparatus 10 for treatment of the eye 13 by using only one hand (not shown), when desired, another hand can also participate in the treatment procedure.

In operation, as shown in FIG. 5A, the patient 22 tilts his/her head backwards to take a horizontal position, and closes the eye under treatment. The operator places the apparatus 10 over the user's face such that the foot 171 of the upper leg 17 is positioned on the user's forehead 24 while the foot 181 of the lower leg 18 is positioned on the user's cheek 25. Then, the operator aligns the nozzle 232 of the dispenser 23 to point it toward the eye 13, and places the eyelid-retaining thread 194 onto the upper eyelid 133 near the eyelashes 134.

Further, the apparatus 10 is pulled up along the user's face 21 in the direction shown by an arrow A, while it remains pressed against the face, such that the upper leg 17 is skidded up on the face, and the eyelid-retaining thread 194, which is stretched over the upper eyelid 133, opens the upper eyelid 133 to expose the eye 13 to the nozzle 232. This state of the eye treatment method is shown in FIG. 5B. The operator then applies a pressure on the lower leg 18 in the direction shown by an arrow B to bend the lower leg 18 inwardly. As shown in FIG. 5C, as a result of this action, the leg part 184 that is positioned on patient's cheek 25 is pushed down along the face together with the cheek 25 and with the lower eyelid 132 in the direction shown by arrow F, thereby opening and exposing a conjunctival sac of the eye 13. The operator then squeezes the bottle of the eye drop dispenser 23 with his/her fingers, as shown by arrows C and D, such that eye treatment medication is dispensed from the nozzle 232 into the eye 13.

Referring to FIGS. 6A through 6E, a method of administration of medication into an eye of a patient by using the apparatus 10 is illustrated, according to another embodiment of the present invention. According to this embodiment, the method can be implemented not only by an operator, but also by the patient himself/herself. Although treatment of the patient's left eye is shown in FIGS. 6A through 6E, generally, the eye can be either the patient's left eye or right eye, mutatis mutandis. Although the user (i.e., operator or patient) can hold the apparatus 10 for treatment of the eye 13 only by using one hand (not shown), when desired, another hand can also participate in the treatment procedure.

In operation, as shown in FIG. 6A, the patient 22 tilts his/her head backward to take a horizontal position and places the apparatus 10 over his/her face such that the foot 171 of the upper leg 17 is positioned on the user's forehead 24, the foot 181 of the lower leg 18 is positioned on the user's cheek 25, and the eyelid-retaining thread 194 is positioned onto the upper eyelid 133 into a groove 135 that corresponds to the upper eyelid sulcus. In this position of the apparatus 10, the nozzle 232 of the dispenser 23 can be aligned to point toward the eye 13 under treatment. Accordingly, this position of the eyelid-retaining thread 194 on the upper eyelid 133 in the groove 135 is used as a reference position. When the patient 22 uses the eye treatment apparatus 10 by himself/herself, the location of the eyelid-retaining thread 194 in the groove 135 can be found by using the patient's sensation. The eye 13 can either be open or closed.

Then, as shown in FIG. 6B, the foot 171 of the upper leg 17 is lifted up together with the eyelid-retaining thread 194 from the user's face 21 by rotating the apparatus 10 around the lower foot 181 in the counterclockwise direction, as shown by an arrow R.

Further, as shown in FIG. 6C, the patient closes the eye 13, and pushes the apparatus 10 in the direction shown by an arrow E to bend the lower leg 18 outwardly to the maximal angle of the bending mechanism 182. Then the patient returns the foot 171 of the upper leg 17 to the forehead 24 while retaining the lower leg 18 as bent, and places the eyelid-retaining thread 194 back on the eyelid 133 to be stretched over the upper eyelid 133. It should be understood that the bending of the lower leg 18 provides decrease of the distance between the feet 171 and 181. Thus, the position of the eyelid-retaining thread 194 in the state shown in FIG. 6C is shifted from its reference position in the groove 135 towards the eyelashes 134 at a predetermined distance. This predetermined distance can, for example, be in the range of 7 mm to 11 mm.

Then, as shown in FIG. 6D, the patient 22 releases the bending mechanism 182 from the bent state by relieving his exertion applied to the lower leg 18. The lower leg 18 returns to unbent state by the elastic member 185, which returns in the unstressed state (also shown in FIG. 3A). In turn, the foot 171 slides up along the forehead 24, while it remains pressed against the patient's face 21, and the eyelid 133 is pulled up by the eyelid-retaining thread 194 stretched over the eyelid 133, thereby exposing the eye 13 to the nozzle 232.

As shown in FIG. 6E, the patient then applies a pressure on the lower leg 18 in the direction shown by an arrow B to bend the lower leg 18 inwardly. As a result of this action, the leg part 184 that is positioned on patient's cheek 25 is pushed down along the face together with the cheek 25 and with the lower eyelid 132 in the direction shown by arrow F, thereby opening and exposing a conjunctival sac of the eye 13. The patient then squeezes the bottle of the eye drop dispenser 23 by using his/her fingers, as shown by arrows C and D, such that eye treatment medication is dispensed from the nozzle 232 into the eye 13.

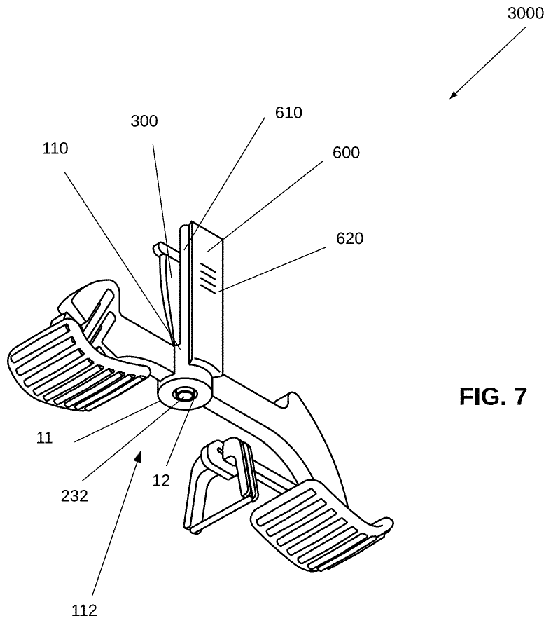

Referring to FIG. 7, an elevated perspective view of an apparatus 3000 for eye treatment is illustrated, according to a further embodiment of the present invention. In this embodiment, the apparatus 3000 includes a supporting platform 11 including a delivery opening 12 having a cylindrical shape. According to this embodiment, an eye drop dispenser 300 suitable for this configuration includes a container in the form of a unit dose vial configured for holding a unit dose of medicament.

The apparatus 3000 also includes a supporting member 600 connected to the lateral side 110 of the supporting platform 11 and adapted for supporting the eye drop dispenser 300 for securing thereof within the delivery opening 12. The supporting member 600 is in the form of a plate having a supporting inner wall 610 attachable to eye drop dispenser 300 when it is placed in the delivery opening 12, and a supporting outer wall 620 configured for gripping by the user's fingers.

The delivery opening 12 is formed within the supporting platform 11 and extends from the top side 111 to the lower side 112 of the supporting platform 11. In this configuration, the delivery opening 12 is configured for holding a nozzle 232 of the eye drop dispenser 300 that is inserted in the delivery opening 12.

As shown in FIG. 7, the delivery opening 12 can be in the form of a cylindrical hole having a predetermined diameter, however, generally, the delivery opening 12 may have any other desired shape suitable to fit the shape and size of the nozzle 232 of the eye drop dispenser 300.

In operation, when the eye drop dispenser 300 is placed in the delivery opening 12, the eye drop dispenser 300 can be squeezed by the user's fingers against the inner wall 610 of the supporting member 600 with a predetermined force, so as to eject the medicament from the vial 300 to the eye 13 of the patient 22.

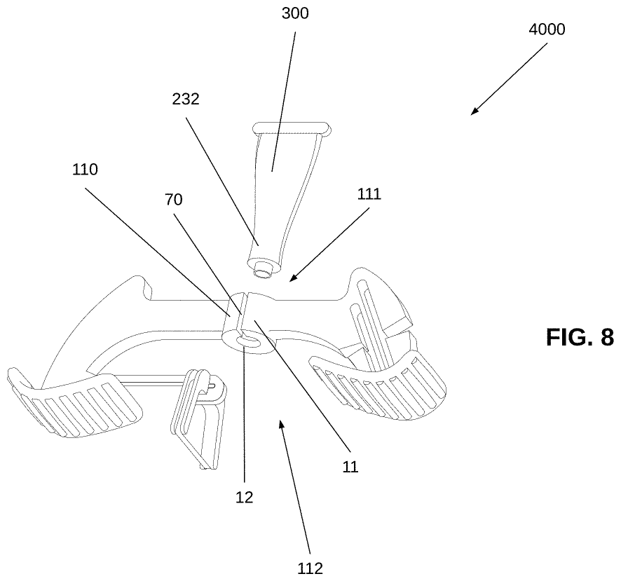

Referring to FIG. 8, an elevated perspective view of an apparatus 4000 for eye treatment is illustrated, according to yet another embodiment of the present invention. In this embodiment, the apparatus 4000 includes a supporting platform 11 including a delivery opening 12 having a cylindrical shape. Although an eye drop dispenser 300 in the form of a unit dose vial is shown in FIG. 8, generally this configuration of the treatment apparatus 4000 can be used with any other eye drop dispenser having a nozzle with a cylindrical or conical shape.

According to this embodiment, the supporting platform 11 is configured for implementing a spring mechanism for gripping the eye drop dispenser 300. Thus, the supporting platform 11 is formed of a pliable and resilient material, such as plastic, enabling the expansion and contraction of the supporting platform 11, thereby varying the cross-sectional dimensions of the delivery opening 12.

As shown in FIG. 8, the supporting platform 11 includes an elongated vertical slit 70 arranged at the lateral side 110 of the supporting platform 11 that extends from the top side 111 to the under side 112 of supporting platform 11. More specifically, the supporting platform 11 is configured for expanding the delivery opening 12 from its normal state to the diameter of the nozzle 232 upon engaging with the eye drop dispenser 300 in order to grasp the nozzle 232. In particular, upon engaging with the eye drop dispenser 300 the delivery opening 12 varies its cross-sectional dimensions to secure eye drop dispenser 300 within the delivery opening.

Those skilled in the art to which the present invention pertains, can appreciate that while the present invention has been described in terms of preferred embodiments, the concept upon which this disclosure is based may readily be utilized as a basis for the designing of other structures, systems and processes for carrying out the several purposes of the present invention.

It should be understood that the apparatus for eye treatment of the present invention is not limited to rinsing and administration of liquid medications. When desired, the supporting platform 11 of the apparatus can be adapted, mutatis mutandis, not only for holding eye drop dispensers, but also for grasping various other medical devices, for example intraocular pressure recording devices, cameras for external ocular photos, fundus photos and other images used in optometry and ophthalmology.

Also, it is to be understood that the phraseology and terminology employed herein are for the purpose of description and should not be regarded as limiting.

Finally, it should be noted that the words “comprising”, “having” and “including” as used throughout the appended claims are to be interpreted to mean “including but not limited to”. It is important, therefore, that the scope of the invention is not construed as being limited by the illustrative embodiments set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. An apparatus for treatment of an eye of a patient by a medication stored in an eye drop dispenser, the apparatus comprising:
a supporting platform including a delivery opening configured for holding an eye drop dispenser therein in the upside-down position.
an upper leg coupled to the supporting platform at one end of the upper leg, and configured for positioning on a forehead above an eyebrow of the patient at the other end of the upper leg;
a lower leg coupled to the supporting platform at one end of the lower leg, and configured for positioning on a top part of a cheek below a lower eyelid; and
an eye opening assembly configured to pull an upper eyelid up for exposing the eye to the eye drop dispenser, and for retaining the eye of the patient in the open position as long as required for the treatment.

2. The apparatus of claim 1, further comprising a locking mechanism integrated with said supporting platform, and configured to provide secure grasping of the eye drop dispenser within the delivery opening.

3. The apparatus of claim 1, further comprising a restricting member configured to limit insertion of the eye drop dispenser into the delivery opening of the supporting platform for holding the eye drop dispenser at a selected distance above the eye of the patient in order to prevent direct contact of the dispenser with the eye.

4. The apparatus of claim 5 wherein the restricting member comprises a through opening having a predetermined dimension adapted to match the dimension of a nozzle of the eye drop dispenser.

5. The apparatus of claim 2, wherein the locking mechanism includes a ratchet mechanism.

6. The apparatus of claim 1, wherein the supporting platform includes an elongated vertical slit arranged at the lateral side of the supporting platform, the slit extending from the top side to the under side of said supporting platform.

7. The apparatus of claim 6, wherein the supporting platform is formed of one or more resilient materials enabling expansion and contraction of the supporting platform for varying the cross-sectional dimensions of the delivery opening upon engaging with the eye drop dispenser to secure it within the delivery opening.

8. The apparatus of claim 1, wherein the supporting platform further comprises a supporting member connected to the lateral side of the supporting platform, the supporting member adapted to secure the eye drop dispenser within the delivery opening.

9. The eye treatment apparatus of claim 1, wherein the upper and lower legs are terminated in feet configured for positioning on the forehead and the cheek of the patient, correspondingly.

10. The eye treatment apparatus of claim 9, wherein the feet are separate elements pivotally attached to the ends of the upper and lower legs, correspondingly.

11. The eye treatment apparatus of claim 1, wherein the lower leg includes a bending mechanism located between the supporting platform and the cushion pad, the bending mechanism is configured to allow the lower leg to be bent into angular form to limited angles having predetermined values outwardly or inwardly with respect to a face of the patient.

12. The eye treatment apparatus of claim 11, wherein the bending mechanism divides the lower leg into two leg parts, manufactured as separately assembled pieces, and connected by an elastic member passing within grooves formed at meeting ends of the leg parts, correspondingly; said leg parts are separated from each other by gaps formed at two sides of the elastic member.

13. The eye treatment apparatus of claim 1, wherein the upper leg and the lower leg are manufactured separately from the supporting platform, and are assembled together with the platform by connecting the upper leg and the lower leg to the upper wall and the lower wall of the platform, correspondingly.

14. The eye treatment apparatus of claim 1, wherein the supporting platform, the upper leg and at least a portion of the lower leg are manufactured as one entire part.

15. The eye treatment apparatus of claim 1, wherein the eye opening assembly includes:
an eyelid pulling frame including two frame arms having distal ends and separated at a predetermined distance;
an eyelid-retaining thread stretched between the distal ends of the frame arms, said eyelid-retaining thread configured to follow an outer contour of the eyeball when the eyelid-retaining thread is placed on the upper eyelid by applying a predetermined pressing force against the eye; and
a spring member configured to provide said predetermined pressing force to the eyelid pulling frame.

16. The eye treatment apparatus of claim 15, wherein the eyelid-retaining thread is a flexible filament having a predetermined strain.

17. The eye treatment apparatus of claim 15, wherein said spring member includes a strip spring.

18. The eye treatment apparatus of claim 15, wherein one end of the spring member is connected to the pulling frame, while the other end of the spring member is connected to the upper leg.

19. The eye treatment apparatus of claim 15, wherein the spring member is connected to the pulling frame at its one end and to the supporting platform at its other end.

20. The apparatus of claim 1, wherein the eye drop dispenser includes a container for holding medicament and a nozzle extending from the container and allowing the medicament to be ejected therefrom.
